(19) United States
(12) Patent Application Publication
(10) Pub. No.: US 2020/0024334 A1
(43) Pub. Date: Jan. 23, 2020

(54) Heterodimeric proteins and preparation method thereof

(71) Applicant: Lei ZHAO, Beijing (CN)

(72) Inventors: Lei Zhao, Beijing (CN); Fan Zhang, Beijing (CN)

(21) Appl. No.: 16/255,482

(22) Filed: Jan. 23, 2019

(30) Foreign Application Priority Data
Jul. 23, 2018 (CN) 201810811940.7

(51) Int. Cl. C07K 16/18; A61P 35/00

(52) U.S. Cl. CPC C07K 16/18; A61P 35/00; C07K 2317/56; C07K 2317/524; C07K 2317/526; C07K 2317/32

(57) ABSTRACT

The present invention provides a heterodimeric protein comprising polypeptides that bind each other containing two CH3 regions, wherein amino acid mutations are introduced into CH3 region of the first polypeptide and CH3 region of the second polypeptide to form pairs of amino acids with polar interactions on their interaction interface, thereby forming a heterodimeric protein specifically. The heterodimeric protein of the present invention can prevent Fc mismatch, avoid homodimer formation, and has high yield and good stability.

### INCORPORATION OF SEQUENCE LISTING

This application contains a sequence listing submitted in Computer Readable Form (CRF). The CFR file containing the sequence listing entitled “PB4082984-SequenceList.txt”, which was created on May 13, 2019, and is 62910 bytes in size. The information in the sequence listing is incorporated herein by reference in its entirety.

### TECHNICAL FIELD

The present invention relates to a heterodimeric protein, comprising individual polypeptides forming the heterodimeric protein and nucleic acid sequences encoding the polypeptides, and also relates to a method of forming the heterodimeric protein.

### BACKGROUND

Antibody-targeted drugs have the advantages of high specificity, minimal side effects and long half-life etc., the treatment method using which is a very promising bio-therapeutic method. At present, the FDA has approved more than 48 antibody drugs for clinical disease treatment. More than 17 antibody drugs have been approved for clinical treatment of tumors, and more antibody drug candidates are undergoing preclinical and clinical research. Nowadays, antibody-targeted drugs have gradually become an important means of clinical treatment of tumors. However, due to the complexity and multi-factor feature of tumorigenesis and development, it is difficult to achieve better efficacy with single-targeted antibodies that rely solely on a single target. Therefore, the vast majority of patients gradually develop tolerance and recurrence during the treatment of cancer. Therefore, there is an urgent need to develop targeted antibodies with better therapeutic effects for clinical disease treatment. Bispecific or multi-specific antibodies with the capacity to target multiple targets exhibit better clinical applications than single-targeted antibodies and currently become a focus in the field of targeted antibody research.

Bispecific/multi-specific antibodies do not exist in nature and can only be prepared by special methods. Blinatumomab and Catumaxomab, two bispecific antibody drugs, have been approved by FDA, which are generated through genetic engineering methods and hybridoma techniques, respectively. However, due to the multiple possible antibody forms produced by the random pairing of the light chain and the heavy chain of bispecific antibodies generated by hybridoma method, it is very difficult to produce and purify these bispecific antibodies. Moreover, the heterogeneous origin and immunogenicity of bispecific antibodies developed by the rat-mouse hybridoma technique have greatly limited their clinical efficacy. Therefore, most of the current bispecific antibody drugs for clinical trials are prepared by genetic engineering techniques. It is well known that the antibody with intact IgG architecture shows many advantages in clinical use, which plays an essential role in induction of antibody-mediated ADCC (antibody-dependent cell-mediated cytotoxicity)/ADCP (antibody-dependent cell-mediated phagocytosis) killing, antibody tumor penetration and half-life of antibody. The Knobs-into-Holes (KIH) technology is currently one of the main techniques for preparing bi-/multi-specific antibodies with IgG-like architectures. The KIH technique is introduced several mutations in the interface of CH3-CH3 domain in the Fc region of antibody. The hydrophobic amino acids with large side chain are introduced in one side of CH3 interface, while the hydrophobic amino acids with small side chain are introduced in another side of CH3 interface, correspondingly. To further stabilize the heterodimer of CH3-CH3, a disulfide bond is introduced into the CH3-CH3 interface. However, in their research results, about 5% of the polypeptides still form homodimers (Brinkmann U, Kontermann R E. The making of bispecific antibodies. MAbs. 2017; 9(2): 182-212.), which are difficult for purification and production in industrial manufacturing process, subsequently.

In order to solve the problems mentioned above, the present invention discloses a method for preparing a heterodimeric protein. The heterodimeric protein involved comprises two polypeptides of the correspondingly modified CH3 domain. The specific interaction between the modified CH3 domains promotes formation of CH3-CH3 heterodimer, preventing the mispairing and formation of CH3-CH3 homodimer.

### SUMMARY OF THE INVENTION

The object of the present invention is to provide a heterodimeric protein.

Another object of the present invention is to provide a method for the preparation of the heterodimeric protein.

In the first aspect of the present invention, a heterodimeric protein is provided, wherein the heterodimeric protein comprises polypeptides that bind each other containing two CH3 regions, and amino acid mutations are introduced into CH3 region of the first polypeptide and CH3 region of the second polypeptide to form pairs of amino acids with polar interactions on their interaction surface and thus form a heterodimeric protein with specific interactions, wherein, the amino acid mutations comprise:

D356K, Q347K and D399K in CH3 region of the first polypeptide and K439D, K360E, K409D and K392D in CH3 region of the second polypeptide; or

D356K, Q347K and D399K in CH3 region of the first polypeptide and K439E, K360E, K409D and K392D in CH3 region of the second polypeptide.

In another preferred embodiment, the amino acid mutations further comprise: K392C in CH3 region of the first polypeptide.

In another preferred embodiment, the amino acid mutations further comprise: D399C in CH3 region of the second polypeptide.

In another preferred embodiment, the amino acid mutations comprise: D356K, Q347K, D399K and K392C in CH3 region of the first polypeptide and K439D, K360E, K409D, K392D and D399C in CH3 region of the second polypeptide.

Alternatively, the amino acid mutations comprise: D356K, Q347K, D399K and K392C in CH3 region of the first polypeptide and K439E, K360E, K409D, K392D and D399C in CH3 region of the second polypeptide.

In another preferred embodiment, the polypeptide pair of the heterodimeric protein is a polypeptide pair selected from the following group:

the first polypeptide of SEQ ID NO: 18 and the second polypeptide of SEQ ID NO: 20 (test1-1);

the first polypeptide of SEQ ID NO: 18 and the second polypeptide of SEQ ID NO: 22 (test1-2);

the first polypeptide of SEQ ID NO: 24 and the second polypeptide of SEQ ID NO: 26 (test1-5); and

the first polypeptide of SEQ ID NO: 24 and the second polypeptide of SEQ ID NO: 28. (test1-6)

In another preferred embodiment, the heterodimeric protein is an antibody protein or a fusion protein.

In another preferred embodiment, the heterodimeric protein is a dual-targeted antibody or a dual-targeted fusion protein.

In another preferred embodiment, the structural type of the heterodimeric protein is one of the following structures:

Y-Shaped Structure Comprising Two Fab/scFv/Fusion Protein (Receptor or Ligand)—CH2-CH3 Chains;

Y-shaped structure comprising two Fab/scFv/fusion protein (receptor or ligand)—CH3 chains;

Y-shaped structure comprising two Fab/scFv/fusion protein (receptor or ligand)—Fab/scFv/fusion protein (receptor or ligand)-CH2-CH3 chain; or

Y-shaped structure comprising two Fab/scFv/fusion protein (receptor or ligand)—Fab/scFv/fusion protein (receptor or ligand)-CH3 chain.

In another preferred embodiment, the heterodimeric protein comprises a disulfide bond formed by amino acid mutations between the CH3 region of the first polypeptide and the CH3 region of the second polypeptide, and the formation of the disulfide bond is due to K392C of the CH3 region of the first polypeptide and D399C of the CH3 region of the second polypeptide.

In another preferred embodiment, the heterodimeric protein comprises anti-tumor antibodies.

In the second aspect of the present invention, a method for producing the heterodimeric protein according to the first aspect of the invention is provided, wherein the amino acid mutations are introduced into CH3 region of the first polypeptide and CH3 region of the second polypeptide to form heterodimer specifically.

In another preferred embodiment, the amino acid to which the amino acid mutation is introduced in the CH3 region is distributed on the interface peripheral regions of the two polypeptides in the protein spatial structure.

In another preferred embodiment, the method utilizes a combination of positive and negative charge interactions formed between the CH3 region of the first polypeptide and the CH3 region of the second polypeptide and the formation of a disulfide bond, to form the dimerization.

In the third aspect of the present invention, a pharmaceutical composition or formulation is provided, wherein the pharmaceutical composition or formulation comprises:

(i) the heterodimeric protein according to the first aspect of the present invention;

(ii) a pharmaceutically acceptable carrier.

In another preferred embodiment, the pharmaceutical composition or formulation is selected from the group consisting of: a suspension formulation, a liquid formulation, or a lyophilized formulation.

In another preferred embodiment, the liquid formulation is an injection formulation.

In another preferred embodiment, the liquid formulation has a shelf life of one to three years, preferably one to two years, more preferably one year.

In another preferred embodiment, the liquid formulation has a storage temperature of from 0° C. to 16° C., preferably from 0° C. to 10° C., more preferably from 2° C. to 8° C.

In another preferred embodiment, the lyophilized formulation has a shelf life of from six months to two years, preferably from six months to one year, more preferably half a year.

In another preferred embodiment, the lyophilized formulation has a storage temperature of ≤42° C., preferably ≤37° C., more preferably ≤30° C.

In another preferred embodiment, the pharmaceutically acceptable carrier comprises: a surfactant, a solution stabilizer, an isotonicity adjusting agent, a buffer, or a combination thereof.

In another preferred embodiment, the solution stabilizer is selected from the group consisting of a saccharide solution stabilizer, an amino acid solution stabilizer, an alcohol solution stabilizer, or a combination thereof.

In another preferred embodiment, the saccharide solution stabilizer is selected from the group consisting of a reducing saccharide solution stabilizer or a non-reducing saccharide solution stabilizer.

In another preferred embodiment, the amino acid solution stabilizer is selected from the group consisting of monosodium glutamate or histidine.

In another preferred embodiment, the alcohol solution stabilizer is selected from the group consisting of tri-alcohols, higher saccharide alcohols, propylene glycol, polyethylene glycols, or combinations thereof.

In another preferred embodiment, the isotonicity adjusting agent is selected from the group consisting of sodium chloride or mannitol.

In another preferred embodiment, the buffer is selected from the group consisting of TRIS, histidine buffer, phosphate buffer, or a combination thereof.

In another preferred embodiment, the pharmaceutical composition or formulation is administered to a human or non-human animal.

In another preferred embodiment, the non-human animal comprises: a rodent (such as a rat, a mouse), a primate (such as a monkey).

In another preferred embodiment, the component (i) is from 0.1% to 99.9% by weight, preferably from 10% to 99.9% by weight, more preferably from 20% to 99.9% by weight, of the total weight of the pharmaceutical composition or formulation.

In another preferred embodiment, the administration of the pharmaceutical composition or formulation is carried out in an amount of from 0.01 g to 10 g per day, preferably from 0.05 g to 5000 mg per day, more preferably from 0.1 g to 3000 mg per day.

In another preferred embodiment, the pharmaceutical composition or formulation is for use in inhibiting and/or treating a tumor.

In another preferred embodiment, the inhibiting and/or treating a tumor comprises a delay associated with the development of symptoms associated with tumor growth and/or a decrease in the severity of such symptoms.

In another preferred embodiment, the inhibiting and/or treating a tumor further comprises a reduction of the pre-existing symptoms accompanying tumor growth and prevention of the appearance of other symptoms.

In another preferred embodiment, the pharmaceutical composition or formulation can be administered in combination with other antitumor agents for the treatment of tumors.

In another preferred embodiment, the antitumor agent co-administered is selected from the group consisting of: a cytotoxic drug, a hormone antiestrogen, a biological response modifier, a monoclonal antibody, or some other drugs currently mechanism unknown and pending further Research.

In another preferred embodiment, the cytotoxic drug comprises: a drug that acts on the chemical structure of DNA, a drug that affects nucleic acid synthesis, a drug that acts on nucleic acid transcription, a drug that acts mainly on tubulin synthesis, or other cytotoxic drugs.

In another preferred embodiment, the drug that acts on the chemical structure of DNA comprises: an alkylating agent such as nitrogen mustard, nitrosour, a methylsulfonate; a platinum compound such as cisplatin, carboplatin, platinum oxalate; Mitomycin (MMC).

In another preferred embodiment, the drug that affects nucleic acid synthesis comprises: dihydrofolate reductase inhibitors such as methotrexate (MTX) and Alimta, and the like; thymidine synthase inhibitors such as fluorouracil (5FU, FT-207, capecitabine) etc.; purine nucleoside synthase inhibitors such as 6-mercaptopurine (6-MP) and 6-TG etc.; nucleoside reductase inhibitors such as hydroxyurea (HU) etc.; DNA polymerase inhibition agents such as cytarabine (Ara-C) and Gemz etc.

In another preferred embodiment, the drug that acts on nucleic acid transcription comprises: a drug that selectively acts on a DNA template, inhibits DNA-dependent RNA polymerase, thereby inhibiting RNA synthesis, such as actinomycin D, daunorubicin, Doxorubicin, epirubicin, aclarithromycin, Clomithromycin, and the like.

In another preferred embodiment, the drug that acts mainly on tubulin synthesis comprises: paclitaxel, taxotere, vinblastine, vinorelbine, podophyllum, homoharringtonine.

In another preferred embodiment, the other cytotoxic agent comprises asparaginase that mainly inhibits synthesis of proteins.

In another preferred embodiment, the hormonal antiestrogens comprise: tamoxifen, droloxifene, exemestane, etc.; aromatase inhibitors: aminoglutethimide, lentaron, letrozole, Anastrozole etc.; antiandrogen: Fluoramide RH-LH agonist/antagonist: Zoladex, enatone and so on.

In another preferred embodiment, the biological response modifier comprises: interferon; interleukin-2; thymosin.

In another preferred embodiment, the monoclonal antibodies comprises: MabThera; Cetuximab (C225); Trastuzumab; Bevacizumab (Avastin); Yervoy (Ipilimumab); Nivolumab (OPDIVO); Pembrolizumab (Keytruda); Atezolizumab (Tecentriq).

In the forth aspect of the present invention, use of the heterodimeric protein according to claim 1 is provided, wherein the use is for the preparation of a medicament for treating a tumor or an antiviral drug.

In the fifth aspect of the present invention, a method of forming a heterodimer between polypeptides comprising a CH3 region is provided, comprising introducing amino acid mutations on the interaction surface of two CH3 regions of the polypeptide to form amino acid pairs having a polar interaction, and to form a specific interacting heterodimeric protein; wherein the amino acids introduced with the amino acid mutations is distributed on the outer structure of the mutual interface of the two polypeptides in the spatial structure of the protein.

It should be understood that, in the present invention, each of the technical features specifically described above and below (such as those in the Examples) can be combined with each other, thereby constituting new or preferred technical solutions which need not be specified again herein.

### DESCRIPTION OF THE DRAWINGS

FIG. 1 is a schematic diagram showing the structure and different regions of an IgG1 antibody.

In the figure, VH is the antibody heavy chain variable region, VL is the antibody light chain variable region, CH1 is the antibody heavy chain constant region 1, CL is the antibody light chain constant region, Hinge is the antibody hinge region, CH2 is the antibody heavy chain constant region 2, and CH3 is the antibody heavy chain constant region 3.

FIG. 2 is a schematic diagram showing the spatial structure of a CH3 heterodimer.

In the CH3 heterodimeric control group a and b, the interaction interface includes the dark part and the light part. The light part indicates the positions of mutation sites on the CH3 heterodimer interaction surface in the CH3 heterodimer control group. In Key1 and Lock1, the dark part of the dark part indicates the CH3 dimer interaction contact surface, and the light gray color indicates the positions of the Test1 mutation sites on the CH3 heterodimer interaction surface.

FIG. 3 shows a schematic diagram of several structural combinations of heterodimeric proteins.

FIG. 3 panel A is polypeptide chains in which an antibody Fab, a single chain antibody (scFv), a receptor protein extramembrane region or a ligand, an antibody hinge or linker and CH2, CH3 are in tandem. The polypeptides form a specific combination by means of a pair of CH3 regions (or fragments) provided in the present invention;

FIG. 3 panel B is polypeptide chains in which an antibody Fab, a single chain antibody (scFv), a receptor protein extramembrane region or a ligand, an antibody hinge or linker and CH3 are in tandem. The polypeptides form a specific combination by means of a pair of CH3 regions (or fragments) provided in the present invention;

FIG. 3 panel C is polypeptide chains in which an antibody Fab, a single chain antibody (scFv), a receptor protein extramembrane region or a ligand, an antibody hinge or linker and CH3 are in tandem. The polypeptides form a specific combination by means of a pair of CH3 regions (or a fragment) provided in the present invention;

FIG. 3 panel D is polypeptide chains in which an antibody Fab, a single chain antibody (scFv), a receptor protein extramembrane region or a ligand link in tandem with a single chain antibody (scFv), a receptor protein extramembrane region or a ligand, an antibody hinge or linker and CH2-CH3 by a linker. The polypeptides form a specific combination by means of a pair of CH3 regions (or fragments) provided in the present invention;

FIG. 3 panel E is polypeptide chains in which an antibody Fab, a single chain antibody (scFv), a receptor protein extramembrane region or a ligand link in tandem with a single chain antibody (scFv), a receptor protein extramembrane region or a ligand, an antibody hinge or linker and CH3 by a linker. The polypeptides form a specific combination by means of a pair of CH3 regions (or fragments) provided in the present invention; FIG. 3 panel F is polypeptide chains in which an antibody Fab, a single chain antibody (scFv), a receptor protein extramembrane region or a ligand link in tandem with a single chain antibody (scFv), a receptor protein extramembrane region or a ligand, an antibody hinge or linker and CH3 by a linker. The polypeptides form a specific combination by means of a pair of CH3 regions (or fragments) provided in the present invention.

FIG. 4 is a diagram for the mutation patterns of the modified CH3-CH3 heterodimer protein.

The corresponding amino acid sites in the CH3-CH3 wild-type were mutated to the amino acids as shown in the figure, which includes the control group, and the experimental group test1, Test1-2, Test2, Test2-2, Test1-5 and Test1-6. The control group includes the control group a and the control group b; the test 1 includes the key 1 and the lock 1; the test 1-2 includes the key 1 and the lock 1-2; the test 2 includes the key 2 and the lock 2; the test 2-2 includes the KEY 2 and the lock 2-2; Test1-5 includes key1-5 and lock1-5; Test1-6 includes key1-5 and lock1-6.

FIG. 5 is the evaluation results of a molecular dynamics simulation of CH3-CH3 structural stability.

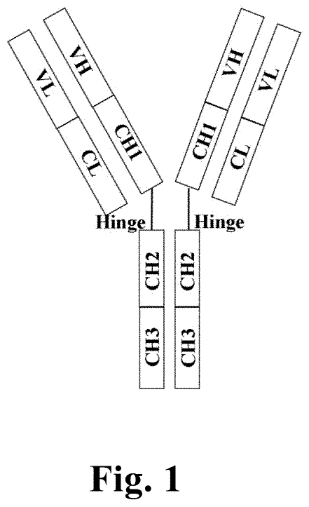

Molecular dynamics simulations were performed on the wt of CH3-CH3 dimer and Test1, Test1-2, Test2, Test2-2 and the control group, at a temperature of 300K and 355K respectively, and on a time scale of 1 microsecond. The variation of the spatial structure of CH3 dimer at different time points was analyzed by means of root mean square offset (RMSD). As shown in the figure, all of the structures can be kept relatively stable at a temperature of 300K, while at a temperature of 355K, WT still maintains structural stability; the structure of the control group fluctuates relatively large; and the experimental group remains relatively stable.

FIG. 6 is a schematic view showing the structure of a test model designed to verify the assembly efficiency of the heterodimeric protein of the present invention.

The left part of the CH3 heterodimeric protein validation model is the intact C225 antibody heavy and light chain combination, and the right part consists of CL replacing the Fab region of the antibody. According to the mutation sites of the present invention, corresponding mutation sites are introduced in the CH3 regions on the left and right sides, respectively. Since the molecular weights of the heavy chains on the left and right sides are significantly different, the heterodimeric protein assembly efficiency can be quickly evaluated.

FIG. 7 is the result of stability of the heterodimeric protein test model;

PD-1 whole antibody, control group and representative experimental group are diluted to 1 μg/ml with PBS. And after 1, 3 and 7 days of incubation in a 37-degree water bath, the stability is analyzed by silver staining after SDS/PAGE.

FIG. 8 is the result of detecting binding of a heterodimeric protein to a target protein EGFR using flow cytometry method;

After EGFR-positive cells are incubated with these test model proteins, they are stained with fluorescent secondary antibodies and their mean fluorescence intensity (MFI) was measured by flow cytometry.

### EMBODIMENTS FOR CARRYING OUT THE INVENTION

The inventors have extensively and intensively studied and, after extensive screening, first unexpectedly developed a heterodimeric protein and a preparation method thereof. The method is to carry out corresponding polarity modification on the interface of the two CH3 regions (or fragments), so that the polypeptides containing the modified CH3 regions could efficiently form a heterodimeric protein, thereby consequently preventing the polypeptides with the modified CH3 regions from formation of a homodimeric protein, and reducing the homomeric mismatch probability. The present invention is completed on this basis.

Heterodimeric Protein

According to one aspect of the present invention, the heterodimeric protein comprises two polypeptides with specific interaction at modified CH3 regions, wherein amino acid mutations are respectively introduced into CH3 region of the first polypeptide and CH3 region of the second polypeptide to form specific interaction through the introduced polar amino acid mutations, thereby forming a heterodimeric protein specifically and efficiently.

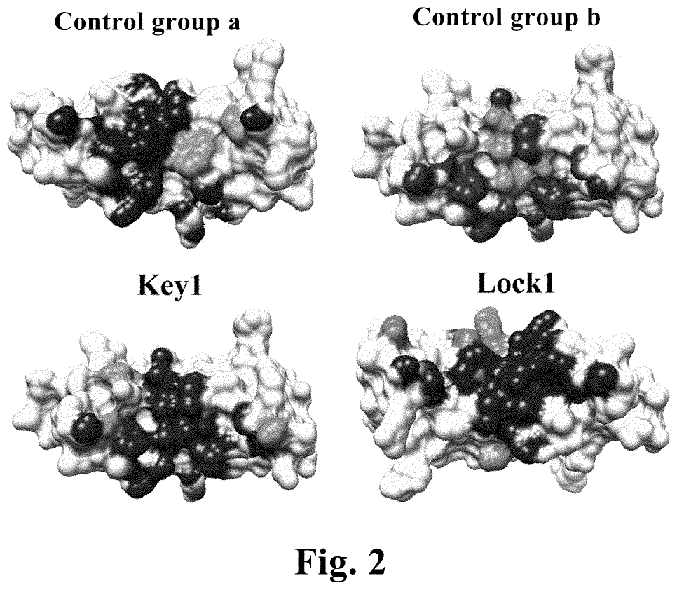

Different from mutating amino acids at the interface of two CH3 regions in Fc to form a heterodimer in the KIH technology, constructing heterodimeric protein in the present invention is started from introducing amino acid mutations in the peripheral region of interface, where the two polypeptides contact with each other, in the spatial structure of the protein (as shown in FIG. 2).

In the present invention, the polypeptides to forming a heterodimer may be any protein comprising complete CH3 regions or partial CH3 regions (or fragments), such as an antibody protein, a fusion protein or the like.

The specific structures of the formed heterodimers are selected from the following structural types:

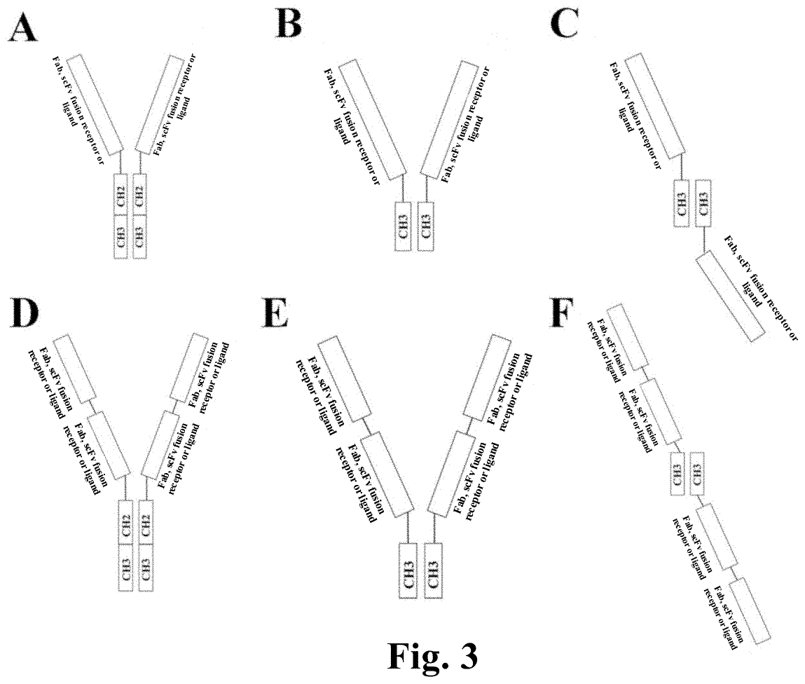

Y-shaped structure comprising two Fab/scFv/fusion receptor or ligand-CH2-CH3 chains (as shown in FIG. 3 panel A);

Y-shaped structure comprising two Fab/scFv/fusion receptor or ligand-CH3 chains (as shown in FIG. 3 panel B and 3 panel C);

Y-shaped structure comprising two Fab/scFv/fusion receptor or ligand-Fab/scFv/fusion receptor or ligand-CH2-CH3 chains (as shown in FIG. 3 panel D);

Y-shaped structure comprising two Fab/scFv/fusion receptor or ligand-Fab/scFv/fusion receptor or ligand-CH3 chains (as shown in FIG. 3 panel E and 3 panel F);

The above structural types are merely exemplary and do not limit the present invention. The skilled in the art will understand that one main feature of the present invention is polar interaction which occurs between the amino acids of the CH3 regions of the two polypeptides. There is no limitation on type of the polypeptide.

Furthermore, in the CH3 region of the first polypeptide (the first CH3 region), amino acids at the selected sites are mutated to positive charged lysines; in the CH3 region of the second polypeptide (the second CH3 region), amino acids at the selected sites are mutated to a negative charged glutamic acid or aspartic acid; optionally, a disulfide bond may be formed by amino acid mutation between the first CH3 region and the second CH3 region to further stabilize the heterdimer.

Preferably, the amino acid mutation sites of the first CH3 region are selected from amino acid positions 356, 347, 399 and 392, and the amino acid mutation sites of the second CH3 are selected from amino acid positions 439, 360, 409, 392 and 399. The positions of the above mutation sites are taken as the reference template by the amino acid numbers of the CH3 region in Atwell S, Ridgway J B B, Wells J A, Carter P. Stable heterodimers from remodeling the domain interface of a homodimer using a phage display library.—PubMed—NCBI. J Mol Biol. 1997; 270(1):26-35.

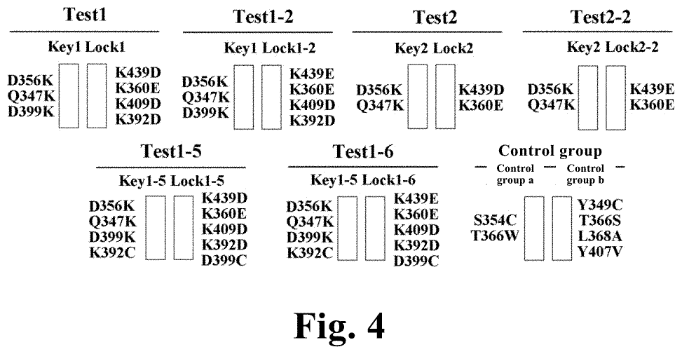

More preferably, the amino acid mutations of the first CH3 region are selected from the group consisting of D356K, Q347K, D399K and K392C; the amino acid mutations of the second CH3 region are selected from K439D, K439E, K360E, K409D, K392D and D339C (see FIG. 4).

According to a preferred embodiment of the present invention, mutations in the first CH3 region of the heterodimeric protein are D356K, Q347K and D399K, and mutations in the second CH3 region are K439D, K360E, K409D and K392D.

According to another preferred embodiment of the present invention, mutations in the first CH3 region of the heterodimeric protein are D356K, Q347K and D399K, and mutations in the second CH3 region are K439E, K360E, K409D and K392D.

According to still another preferred embodiment of the present invention, mutations in the first CH3 region of the heterodimeric protein are D356K, Q347K, D399K and K392C, and mutations in the second CH3 region are K439E, K360E, K409D, K392D and D399C.

The heterodimeric protein may be a dual targeting antibody or a dual targeting fusion protein.

Formulation comprising a heterodimeric protein and the administration thereof

The heterodimeric protein can form a pharmaceutical formulation together with a pharmaceutically acceptable ingredients to exert a more stable therapeutic effect, and these formulations can ensure the structural integrity of the core amino acid sequence of the heterodimeric protein in the present invention, and at the same time ensure the multiple functional groups of the protein protected against degradation (including but not limited to coagulation, deamination or oxidation). The formulation may be in various forms. Generally, liquid formulations are typically stable for at least one year at 2° C. to 8° C. and lyophilized formulations are stable for at least six months at 30° C. The formulations may be suspension, aqueous injection solution, or lyophilized formulation, etc., which are commonly used in the pharmaceutical field, wherein aqueous solution or lyophilized formulation is preferred.

For the aqueous injection solution or lyophilized formulation of the heterodimeric protein of the present invention, the pharmaceutically acceptable ingredient includes one of surfactants, solution stabilizers, isotonicity adjusting agents and buffer solutions, or a combination thereof, wherein the surfactants include nonionic surfactants such as polyoxyethylene sorbitan fatty acid esters (Tween 20 or 80); poloxamer (such as poloxamer 188); Triton; sodium dodecyl sulfate (SDS); sodium lauryl sulfate; tetradecyl, linoleyl or octadecyl sarcosine; Pluronics; MONAQUAT™, etc., the addition amount of which should minimize the granulation tendency of the heterodimeric protein. The solution stabilizer may be a sugar including reducing sugar and non-reducing sugaror, amino acids include monosodium glutamate or histidine, alcohols include one of a trihydroxy alcohol, a higher sugar alcohol, a propylene glycol, and a polyethylene glycol or a combination thereof. The solution stabilizer is added in an amount such that the final formed formulation remains stable for a period of time that is considered stable by those skilled in the art. The isoosmotic adjusting agent may be one of sodium chloride and mannitol, and the buffer may be one of TRIS, histidine buffer, and phosphate buffer.

When the heterodimeric protein or the pharmaceutical formulation thereof is administered to animals including human, the dosage is different depending on the age and weight of the patient, the disease characteristics and severity, and the administration route, and which can refer to the results and various conditions of an animal experiment. The total dose can not exceed a certain range. Specifically, the dose for intravenous injection is 0.1 to 3000 mg/day.

The heterodimeric protein of the present invention and a pharmaceutical preparation containing the same can be used as an anti-tumor drug for tumor treatment. The term “anti-tumor drug” as used in the present invention refers to a drug able to inhibit and/or treat tumor, the effect of which may include a delay of symptoms accompanying the development associated with tumor growth and/or a decrease in the severity of these symptoms, and further include a decreased symptom accompanying tumor growth which already exists and the prevention of other symptoms, and also reduce or prevent metastasis.

The heterodimeric protein and the pharmaceutical formulation thereof in the present invention can also be administered for the treatment of tumors in combination with other anti-tumor drugs, wherein the anti-tumor drugs used in combination include but not limited to: 1. Cytotoxic drugs (1) Drugs acting on the chemical structure of DNA: alkylating agents such as nitrogen mustards, nitrosoureas, methyl sulfonates; platinum compounds such as cisplatin, carboplatin and Oxaliplatin and the like; mitomycin (MMC); (2) Drugs affecting nucleic acid synthesis: dihydrofolate reductase inhibitors such as methotrexate (MTX) and Alimta, etc; thymidine synthase inhibitors such as fluorouracil (SFU, FT-207, capecitabine), etc.; purine nucleoside synthase inhibitors such as 6-mercaptopurine (6-MP) and 6-TG, etc.; nucleoside reductase inhibitors such as hydroxyurea (HU), etc.; DNA polymerase inhibitors such as cytarabine (Ara-C) and Gemzar, etc.; (3) Drugs that act on nucleic acid transcription: drugs that selectively act on DNA templates and inhibit DNA-dependent RNA polymerase, thereby inhibiting RNA synthesis, such as actinomycin D, daunorubicin, doxorubicin, epirubicin, aclarubicin, mithramycin, etc.; (4) Drugs mainly acting on tubulin synthesis: paclitaxel, taxotere, vinblastine, vinorelbine, podophyllum, homoharringtonine; (5) Other cytotoxic drugs: Asparaginase mainly inhibiting protein synthesis; 2. hormone: (1) anti-estrogen: tamoxifen, droloxifene, exemestane, etc.; (2) aromatase inhibitors: aminoglutethimide, lentaron, letrozole, anastrozole, etc.; anti-androgen: flutamide; RH-LH agonist/antagonist: zoladex, enatone, etc.; 3. biological response modifier: tumor interferon is inhibited mainly through the body's immune function; interleukin-2; thymosin; 4. monoclonal antibodies: Rituximab (MabThera); Cetuximab (C225); Herceptin (Trastuzumab); Bevacizumab (Avastin); Yervoy (Ipilimumab); Nivolumab (OPDIVO); Pembrolizumab (Keytruda); Atezolizumab (Tecentriq); 5. Other drugs that the mechanismis currently unknown and need to be further studied: (1) cell differentiation inducers such as retinoids; (2) apoptosis inducers.

Preparation Method

According to another aspect of the present invention, it provides a method of forming a heterodimer between polypeptides having a CH3 region, in which the polar amino acids are introduced in the interaction region of CH3-CH3 to specifically and efficiently form the heterodimer protein. Preferably, the interaction interface between two CH3 regions (or CH3-CH3 regions) where the amino acid mutations are introduced is spatially located in the peripheral region of the contact interface between two polypeptides.

According to another aspect of the present invention, a method for producing the heterodimeric protein is specifically established.

In the method for producing the heterodimeric protein of the present invention, any suitable carrier can be used, which may be one of pDR1, pcDNA3.1 (+), pcDNA3.1/ZEO(+) or pDHFR. And the expression vector includes fusion DNA sequences which are linked to suitable transcriptional and translational regulatory sequences.

Eukaryotic/prokaryotic host cells can be used for the expression of the heterodimeric protein of the present invention. The eukaryotic host cell is preferably a mammalian or insect host cell culture system, preferably cells such as COS, CHO, NSO, sf9 and sf21 etc., and prokaryotic host cell is preferably one of DH5a, BL21 (DE3), and TG1.

The host cell is cultured in the expression condition of heterodimeric protein, thereby the heterodimeric protein can be expressed, isolated and purified.

The heterodimeric protein disclosed in the present invention can be isolated and purified by affinity chromatography. According to the characteristics of the affinity column utilized, conventional methods such as high salt buffer solution, pH changing, etc. can be used to elute the heterodimeric protein binding to the affinity column.

Using the above method, the heterodimeric protein can be purified to a substantially homogeneous substance, such as a single band on SDS-PAGE.

To validate the CH3 heterodimeric proteins as described herein, the inventors have devised a validation model to evaluate of the correct assembling rate of CH3 heterodimeric protein (FIG. 6). As shown in FIG. 6, the left part is the intact antibody heavy and light chain combination; in order to investigate the correct assembling rate of heterodimer protein through molecular weight, the inventors replaced the antigen binding region of the antibody (Fab) with CL in the right part. The advantage of the validation model is that the molecular weight of the heterodimer protein is significantly different with the homodimer mis-paired protein. Thus, the correct assembling rate of CH3 heterodimer protein could be explored quickly through SDS/PAGE.

Specifically, the method of producing the heterodimeric protein comprises the following steps:

1) cloning the variable region gene of the antibody;

2) fusing the antibody heavy chain variable region gene to the human antibody IgG1 antibody CH1 and Fc region to construct a first antibody C225VH-CH1-Hinge-CH2-CH3 fusion fragment; (In the present invention, C225VH refers to the heavy chain variable region of C225 antibody; CH1 refers to the heavy chain constant region 1 of the antibody; Hinge refers to the hinge region of the antibody; CH2 and CH3 are heavy chain constant regions 2 and 3 of the antibody, respectively.)

The first antibody light chain variable region gene is fused with human antibody CL to construct a first antibody C225VL-CL fusion fragment; (In the present invention, C225VL refers to the light chain variable region of C225 antibody, CL refers to the light chain constant region.)

The CL is fused to the Fc region of the IgG1 antibody to construct a CL-Hinge-CH2-CH3 fusion fragment;

3) Constructing a mutant of the CH3 region of the first antibody and the CH3 region of the second antibody, respectively, and the mutation mode is selected from the following (see FIG. 4): D356K, Q347K and D399K in the first CH3 region, and K439D, K360E, K409D and K392D in the second CH3 region;

or D356K, Q347K and D399K in the first CH3 region and K439E, K360E, K409D and K392D in the second CH3 region;

or D356K, Q347K, D399K and K392C in the first CH3 region and K439D, K360E, K409D, K392D and D399C in the second CH3 region;

or D356K, Q347K, D399K and K392C in the first CH3 region and K439E, K360E, K409D, K392D and D399C in the second CH3 region.

4) The fusion gene C225VH-CH1-Hinge-CH2-CH3 containing the first CH3 region mutant, C225VL-CL containing the first CH3 region mutant and the fusion gene CL-Hinge-CH2-CH3 containing the second CH3 region mutant were respectively loaded into an expression vector;

5) The fusion gene C225VH-CH1-Hinge-CH2-CH3, C225VL-CL, CL-Hinge-CH2-CH3 loaded into the expression vector are co-transfection and co-expression, and the heterodimeric protein is obtained by isolation and purification; wherein the expression vector is pcDNA3.1(+) (product of Invitrogen), and is transfected into 293F cells (Thermo Fisher) by PEI method; cells are cultured in serum-free medium for 9 days. And then the heterodimeric protein is purified and obtained from the supernatant of the culture using Protein A chromatography column by affinity chromatography.

The main advantages of the present invention includes that the present invention provides a heterodimeric anti-protein and a method for the preparation thereof. The method is to carry out corresponding polarity modification in the interface periphery regions of CH3-CH3, so that the polypeptides containing the modified CH3 region/fragment could generate a heterodimeric protein, which could effectively prevent the formation of homodimeric protein and reduce the mis-pairing of proteins.

The present invention will be further illustrated below with reference to the specific examples. It should be understood that these examples are only to illustrate the invention but not to limit the scope of the invention. The experimental methods in the following examples which do not specify the specific conditions are usually in accordance with conventional conditions, such as the conditions described in Sambrook et al., Molecular Cloning: Laboratory Manual (New York: Cold Spring Harbor Laboratory Press, 1989), or in accordance with the conditions recommended by the manufacturer. Unless indicated otherwise, parts and percentage are weight parts and weight percentage.

The materials and reagents used in the examples are all commercially available unless otherwise stated.

The following examples illustrate the construction of heterodimers with C225 mAb and CL-Hinge-CH2-CH3 fusion protein. Accordingly, the skilled in the art can select other antibodies or proteins as the first polypeptide and the second polypeptide to form heterodimer of invention.

### Example 1: Cloning of the First Antibody Variable Region

The C225 heavy chain variable region gene and the light chain variable region gene were synthesized according to the patent (PCT/US1996/009847) and designated C225VH and C225VL, respectively. Amino acid sequence of the antibody signal peptide is MGWSCIILFLVATATGVHS. SEQ ID NO: 2 shows the amino acid sequence of the C225 heavy chain variable region, the nucleotide sequence of which is SEQ ID NO: 1; SEQ ID NO: 4 shows the amino acid sequence of the C225 light chain variable region, the nucleotide sequence of which is SEQ ID NO: 3.

### Example 2: Cloning of Human IgG1 Antibody CL, Heavy Chain CH1 and Fc Region

Healthy human lymphocytes were isolated using lymphocyte separation solution (product from Shenggong Biological Engineering Co., Ltd.), and total RNA was extracted using Trizol reagent (product from Life Technologies). The genes of the antibody light chain constant region, heavy chain constant region CH1 and Fc region were amplified by RT-PCR reaction, according to literature (Cloned human and mouse kappa immunoglobulin constant and J region genes conserve homology in functional segments. Hieter P A, Max E E, Seidman J G, Maizel J V Jr, Leder P. Cell. 1980 November; 22(1 Pt 1):197-207.) and literature (The nucleotide sequence of a human immunoglobulin C gammal gene. Ellison J W, Berson B J, Hood L E. Nucleic Acids Res. 1982 Jul. 10; 10(13):4071-9.) The signal peptide gene is ATGGGATGGTCATGTATCATCCTTTTTCTAGTAGCAACTGCAACCGGTGTACA TTCC (SEQ ID No: 41). The PCR product was purified and collected using agarose gel electrophoresis and cloned into pGEM-T vector. After sequencing, it was confirmed that the correct clone was obtained. The CL nucleotide sequence was SEQ ID NO: 5, and the amino acid sequence thereof was SEQ ID NO: 6; the Fc nucleotide sequence was SEQ ID NO: 7, and the amino acid sequence thereof was SEQ ID NO: 8; the CH1 nucleotide sequence was SEQ ID NO: 9, and the amino acid sequence of which was SEQ ID NO: 10.

### Example 3: Construction of Fusion Protein Gene Fragments

The gene fragments obtained in Examples 1 and 2 were fused by Overlap PCR. The antibody heavy chain variable region C225VH, IgG1 antibody CH1 and Fc region cloned in Example 1 were fused to form C225VH-CH1-Hinge-CH2-CH3 fusion fragment; the antibody light chain variable region VL cloned in Example 1 was fused with the light chain constant region cloned in Example 2 to form a C225VL-CL fusion fragment; the CL and Fc genes cloned in Example 2 were fused to form CL-Hinge-CH2-CH3. The PCR product was purified and collected using agarose gel electrophoresis and cloned into pGEM-T vector. After sequencing, it was confirmed that the correct clone was obtained and loaded in to the expression vector. The nucleotide sequence of C225VH-CH1-Hinge-CH2-CH3 was SEQ ID NO: 11, and the amino acid sequence thereof was SEQ ID NO: 12; the nucleotide sequence of C225VL-CL was SEQ ID NO: 13, and the amino acid sequence thereof was SEQ ID NO: 14; the nucleotide sequence of CL-Hinge-CH2-CH3 was SEQ ID NO: 15, and the amino acid sequence of which was SEQ ID NO: 16.

### Example 4: Modification of the Antibody CH3 Regions

The CH3 in the Fc region obtained in Example 2 was modified, and point mutations were introduced into the CH3 regions by using a rapid site-directed mutagenesis kit (TIANGEN). The amino acid sequence numbers of the CH3 region was according to Atwell S, Ridgway J B B, Wells J A, Carter P. Stable heterodimers from remodeling the domain interface of a homodimer using a phage display library.—PubMed—NCBI. J Mol Biol. 1997; 270(1):26-35.

4.1 Preferred Mutation Mode I (Test1)

The first CH3 region mutations are D356K, Q347K and D399K, named Key1; the second CH3 region (CL-Hinge-CH2-CH3) mutations are K439D, K360E, K409D and K392D, named Lock1; wherein the nucleotide sequence of Key1 was SEQ ID NO: 17, and the amino acid sequence thereof was SEQ ID NO: 18; the nucleotide sequence of Lock 1 was SEQ ID NO: 19, and the amino acid sequence thereof was SEQ ID NO: 20;

4.2 Preferred Mutation Mode II (Test1-2)

The first CH3 region mutations are D356K, Q347K and D399K, named Key1; the second CH3 region mutations are K439E, K360E, K409D and K392D, named Lock1-2; wherein the nucleotide sequence of Lock1-2 was SEQ ID NO: 21, and the amino acid sequence thereof was SEQ ID NO: 22;

4.3 Preferred Mutation Mode III (Test1-5)

The first CH3 region mutations are D356K, Q347K, D399K and K392C, named Key1-5; the second CH3 region mutations are K439D, K360E, K409D, K392D and D399C, named Lock1-5; wherein the nucleotide sequence of Key1-5 was SEQ ID NO: 23, and the amino acid sequence thereof was SEQ ID NO: 24; the nucleotide sequence of Lock1-5 was SEQ ID NO: 25, and the amino acid sequence thereof was SEQ ID NO: 26;

4.4 Preferred Mutation Mode IV (Test1-6)

The first CH3 region mutations are D356K, Q347K, D399K and K392C, named Key1-5; the second CH3 region mutations are K439E, K360E, K409D, K392D and D399C, named Lock1-6; wherein the nucleotide sequence of Lock1-6 was SEQ ID NO: 27, and the amino acid sequence thereof was SEQ ID NO: 28.

### Example 5: Construction of the Heterodimeric Fusion Proteins

5.1 Construction of Heterodimeric Protein I

Referring to the preferred mutation mode I of Example 4, corresponding point mutations were introduced in the CH3 region of the fusion protein using a rapid site-directed mutagenesis kit (KM101 from TIANGEN). The Key1 point mutations were introduced into the CH3 region of C225VH-CH1-Hinge-CH2-CH3 and named as C225VH-CH1-Hinge-CH2-CH3-Key1; The Lock1 point mutations were introduced into the CH3 region of CL-Hinge-CH2-CH3 and named as CL-Hinge-CH2-CH3-Lock1. The nucleotide sequence of C225VH-CH1-Hinge-CH2-CH3-Key1 was SEQ ID NO:29, and the amino acid sequence thereof was SEQ ID NO:30; the nucleotide sequence of CL-Hinge-CH2-CH3-Lock1 was SEQ ID NO: 31, and the amino acid sequence thereof was SEQ ID NO:32.

5.2 Construction of Heterodimeric Protein II-IV

Heterodimeric proteins II-IV comprising mutation modes II-IV were constructed according to the above method. The nucleotide sequence of CL-Hinge-CH2-CH3-Lock1-2 was SEQ ID NO: 33, and the amino acid sequence thereof was SEQ ID NO: 34; the nucleotide sequence of C225VH-CH1-Hinge-CH2-CH3-Key1-5 was SEQ ID NO: 35, and the amino acid sequence thereof was SEQ ID NO: 36; the nucleotide sequence of CL-Hinge-CH2-CH3-Lock1-5 was SEQ ID NO: 37, and the amino acid sequence thereof was SEQ ID NO: 38; the nucleotide sequence of CL-Hinge-CH2-CH3-Lock1-6 was SEQ ID NO: 39, and the amino acid sequence thereof was SEQ ID NO: 40.

### Example 6: Expression and Purification of the Heterodimeric Proteins

293F cells (from Thermo Fisher) were cultured in 1 L flasks and transfected at a density of 2×10⁶: heterodimeric protein I (SEQ ID NO: 13, 29, 31), II (SEQ ID NO: 13, 29, 33), III (SEQ ID NO: 13, 35, 37), IV (SEQ ID NO: 13, 35, 39) were dissolved with PEI (Sigma) in a mass ratio of 1:1:1 in 500 μl of serum-free medium (Gibco® FreeStyle™ 293 Expression Medium) respectively, at room temperature for 5 minutes; then were mixed with PEI (Sigma) at room temperature for 20 minutes to form DNA-PEI complex; and then the DNA-PEI mixture was added to the culture flask containing 293F cell suspension. Cell culture supernatants were collected to detect the expression of heterodimeric protein by using ELISA. Goat-anti-human IgG (Fc) was coated on ELISA plate, overnight at 4° C., and the plate was blocked with 2% BSA-PBS at 37° C. for 2 h. Then the supernatant of the resistant clones to be tested or a standard product (Human myeloma IgG1, κ) were added and the plate was incubated at 37° C. for 2 h. Next, HRP-goat-anti-human IgG (κ) was added for binding reaction, and the plate was incubated at 37° C. for 1 h. TMB was added and the plate was incubated at 37° C. for 5 min. And finally, the reaction was terminated by H₂SO₄ and the A450 value was measured. The high expression clones obtained by the screening were expanded and cultured in a serum-free medium. The dual-targeted (or bi-specific) antibody was isolated and purified by using a Protein A affinity column (commercially available from GE). The purified antibody was dialyzed in PBS, and finally the concentration of the purified antibody was quantitatively determined by ultraviolet absorption.

### Example 7: The Evaluation of the CH3-CH3 Structural Stability by Molecular Dynamics Simulation

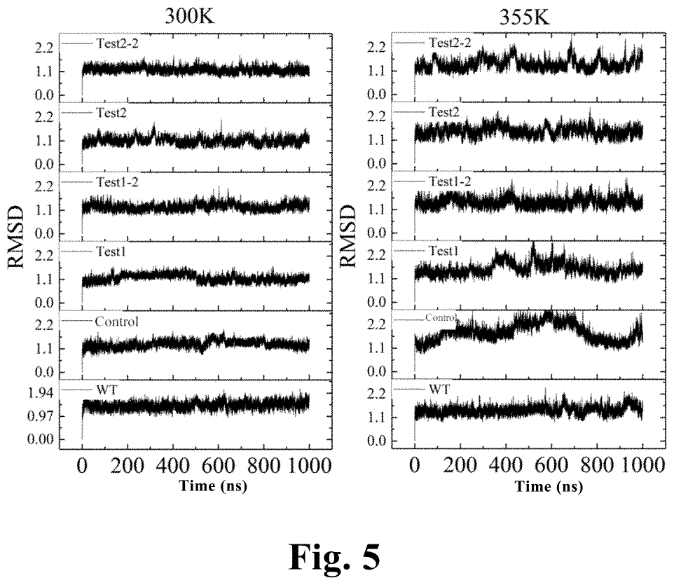

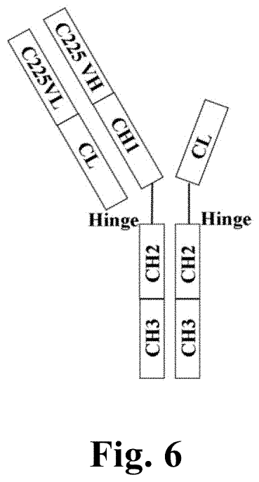

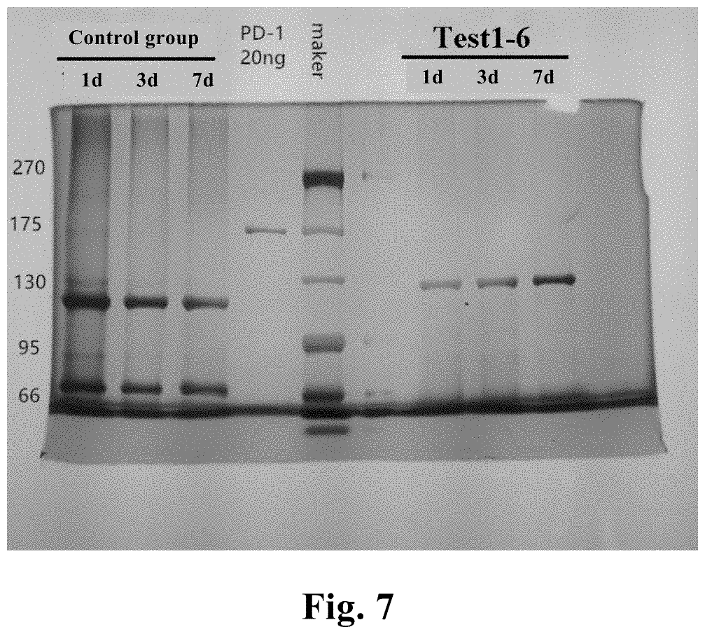

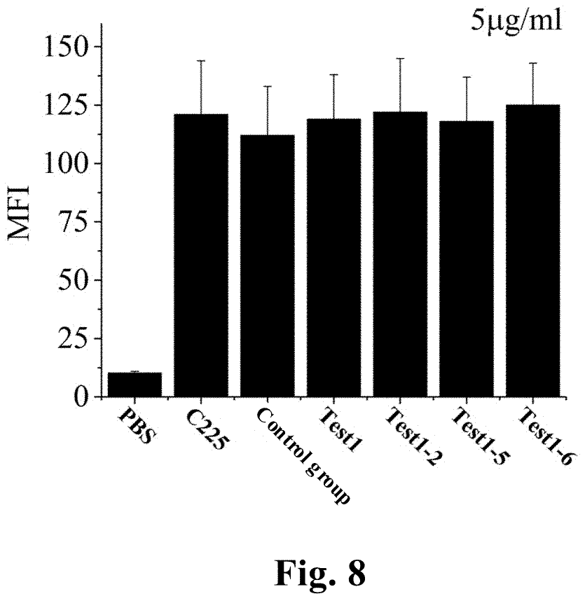

Specific methods are according to [Computer-aided design of new EGFR and PD-1 dual-targeted antibody design and preliminary identification—Master's thesis—Cui Yueqian]. Briefly, after introduction point mutations mentioned above into the CH3 crystal structure (SHSF) to generate test1, test1-2, test1-5, test1-6 structures, these structures and the control group structure (5DI8) were pretreated with pdb4amber to remove water and other ions. TIP3PBOX water molecules with a radius of 12 Å were added around the protein, and the Na⁺ or Cl⁻ ion were added to the system to neutralize the charge of the whole simulation system. After minimizing the energy of the whole simulation system, the NVT system (ntb=1, ntp=0) was used to raise the temperature of the protein system from 100K to 300K or 355K, wherein Langevin dynamics (gamma_ln=1.0) was performed to control the system temperature; after equilibrium of the system by using NPT system (ntb=2, ntp=1), a 1 microsecond time-scale molecular dynamic simulation was performed by employing NPT system (ntb=2, ntp=1), wherein Monte Carlo barostat method and weak-coupling algorithm method were used to control the pressure and temperature, separately. The 1 microsecond time-scale molecular dynamic simulation was further evaluated by RMSD analysis. As shown in FIG. 5, at the temperature of 300K, WT, control group, test1, test1-2, test2, and test2-2 without any large structure fluctuation exhibited relatively stable. However, at 355K, WT, test1, test2, and test2-2 showed relatively stable, while the large fluctuations can be observed in the control group, and test1-2 showed some fluctuations, suggesting that WT, test1, test1-2, and test2, and test2-2 have relatively better structure stability.

### Example 8: Detection of the Assembly Efficiency of the Heterodimeric Proteins

The obtained heterodimeric proteins were analyzed by SDS-PAGE electrophoresis and silver dyeing to evaluate the correct assembly rates of the heterodimeric proteins. As shown in FIG. 6, because of a large molecular weight difference of C225VH-CH1-Hinge-CH2-CH3 and CL-Hinge-CH2-CH3 in the validation model of the present invention, the correct assembling rates of these heterodimeric proteins can be quickly determined. The results showed that test1, test1-2, test1-5 and test1-6 were able to form heterodimers with relatively high correct assembling rates (Table 1).

TABLE 1
Yields of heterodimers derived from CH3 variants

| | heterodimeric protein | | yield |
|---|---|---|---|
| variant | subunit A | subunit B | (%) |
| WT | — | — | 52 ± 2 |
| Control | S354C T366W | Y349C T366S | 85 ± 3 |
| | | L368A Y407V | |
| Test1-1 | D356K Q347K | K439D K360E | 93 ± 3 |
| | D399K | K409D K392D | |
| Test1-2 | D356K Q347K | K439E K360E | 90 ± 3 |
| | D399K | K409D K392D | |
| Test1-5 | D356K Q347K | K439D K360E | 99 ± 1 |
| | D399K K392C | K409D K392D | |
| | | D399C | |
| Test1-6 | D356K Q347K | K439E K360E | 97 ± 0.5 |
| | D399K K392C | K409D K392D | |
| | | D399C | |
| Test2 | D356K Q347K | K439D K360E | 80 ± 2 |
| Test2-2 | D356K Q347K | K439E K360E | 85 ± 4 |

### Example 9: Stability Assay of the Heterodimeric Proteins

The control group and the experimental groups were incubated in PBS solution at a concentration of 5 ug/ml for 1, 3, and 7 days at 37 degrees, and then subjected to SDS-PAGE and silver staining, according to the method in Zhao L, Tong Q, Qian W, et al. Eradication of non-Hodgkin lymphoma through the induction of tumor-specific T cell immunity by CD20-Flex BiFP. Blood. 2013; 122(26):4230-4236. Due to the size limitations of the PAGE gel, the control group and the representative test 1-6 were showed together in the same gel. As shown in FIG. 7, test 1-6 remains stable at day 1, 3, and 7 without observed degradation. More importantly, test 1-6 remained heterodimer at all times and no dissociation of heterodimers was detected. No significant degradation was observed in other experimental groups. The mis-pairing protein in the control group can be detected at dayl, 3 and 7.

### Example 10: Binding Activity Assay of the Heterodimeric Proteins

Flow cytometry was used to detect the binding of the heterodimeric proteins to the target protein EGFR. See Zhao L, Tong Q, Qian W, et al. Eradication of non-Hodgkin lymphoma through the induction of tumor-specific T cell immunity by CD20-Flex BiFP. Blood. 2013; 122(26):4230-4236. Briefly, 2×10⁴ A549 cells (ATCC CCL-185) were incubated with different concentrations of Cetuximab, control group, Test1, Test1-2, Test1-5, and Test1-6, respectively, and washed three times with PBS, and was incubated on ice for 1 hour with the fluorescently labeled secondary antibody against human H+L (Thermo Fisher, A-11013). Flow cytometry was performed after cells were washed with PBS for 3 times. As shown in FIG. 8, the heterodimeric proteins Test1, Test1-2, Test1-5, Test1-6 showed similar binding activity to the parent antibody Cetuximab.

All literatures mentioned in the present application are incorporated herein by reference, as though each one is individually incorporated by reference. Additionally, it should be understood that after reading the above materials, the technicians in the field could make various changes and modifications to the present invention. These equivalents also fall within the scope defined by the appended claims.

1. A heterodimeric protein, which comprises two polypeptides that bind each other through their CH3 regions, wherein mutations of polar amino acid are introduced into CH3 region of a first polypeptide and CH3 region of a second polypeptide to form pairs of amino acids with polar interactions on their interaction interface, thereby forming a heterodimeric protein specifically, wherein the amino acid mutations comprise:
D356K, Q347K and D399K in CH3 region of the first polypeptide, and K439D, K360E, K409D and K392D in CH3 region of the second polypeptide; or
D356K, Q347K and D399K in CH3 region of the first polypeptide, and K439E, K360E, K409D and K392D in CH3 region of the second polypeptide.

2. The heterodimeric protein of claim 1, wherein the amino acid mutations further comprise: K392C in CH3 region of the first polypeptide.

3. The heterodimeric protein of claim 1, wherein the amino acid mutations further comprise: D399C in CH3 region of the second polypeptide.

4. The heterodimeric protein of claim 1, wherein the amino acid mutations comprise: D356K, Q347K, D399K and K392C in CH3 region of the first polypeptide and K439D, K360E, K409D, K392D and D399C in CH3 region of the second polypeptide;
or the amino acid mutations comprise: D356K, Q347K, D399K and K392C in CH3 region of the first polypeptide and K439E, K360E, K409D, K392D and D399C in CH3 region of the second polypeptide.

5. The heterodimeric protein of claim 1, wherein the polypeptide pair of the heterodimeric protein is one of the polypeptide pair selected from the following group:
the first polypeptide of SEQ ID NO: 18 and the second polypeptide of SEQ ID NO: 20(test1-1);
the first polypeptide of SEQ ID NO: 18 and the second polypeptide of SEQ ID NO: 22 (test1-2);
the first polypeptide of SEQ ID NO: 24 and the second polypeptide of SEQ ID NO: 26 (test1-5); and
the first polypeptide of SEQ ID NO: 24 and the second polypeptide of SEQ ID NO: 28 (test1-6).

6. A method for producing the heterodimeric protein according to claim 1, wherein the amino acid mutations are introduced into CH3 region of the first polypeptide and CH3 region of the second polypeptide to form pairs of amino acids with polar interactions on their interaction surface.

7. A pharmaceutical composition or formulation, wherein the pharmaceutical composition or formulation comprises
(i) the heterodimeric protein according to claim 1;
(ii) a pharmaceutically acceptable carrier.

8. A heterodimeric protein which comprises two polypeptides that bind each other through their CH3 regions, wherein mutations of polar amino acid are introduced into CH3 region of a first polypeptide and CH3 region of a second polypeptide to form pairs of amino acids with polar interactions on their interaction interface, thereby forming a heterodimeric protein specifically.

9. The heterodimeric protein of claim 8, which comprises an amino acid mutation pair selected from the group consisting of:
1) D356K, Q347K and D399K in the first CH3 region, and K439D, K360E, K409D and K392D in the second CH3 region;
2) D356K, Q347K and D399K in the first CH3 region and K439E, K360E, K409D and K392D in the second CH3 region;
3) D356K, Q347K, D399K and K392C in the first CH3 region and K439D, K360E, K409D, K392D and D399C in the second CH3 region;
4) D356K, Q347K, D399K and K392C in the first CH3 region and K439E, K360E, K409D, K392D and D399C in the second CH3 region.

10. A method of treating cancer or anti-virus, comprising: administering the heterodimeric protein of claim 1 to a subject in need.


SEQUENCE LISTING (41 sequences)

SEQ ID NO: 1 - DNA, 357 bp, artificial sequence
  synthesized
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctgca

SEQ ID NO: 2 - protein, 119 aa, artificial sequence
  synthesized
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 3 - DNA, 321 bp, artificial sequence
  synthesized
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggatcccttccaggtttagtggcagtggatcagggacagattttactcttagcatcaacagtgtggagtctgaagatattgcagattattactgtcaacaaaataataactggccaaccacgttcggtgctgggaccaagctggagctgaaa

SEQ ID NO: 4 - protein, 107 aa, artificial sequence
  synthesized
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 5 - DNA, 321 bp, artificial sequence
  synthesized
cgtacggtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatgc

SEQ ID NO: 6 - protein, 105 aa, artificial sequence [sequence omitted]

SEQ ID NO: 7 - DNA, 990 bp, artificial sequence
  synthesized
gctagcaccaagggcccatcggtcttccccctggcaccctcctccaagagcacctctgggggcacagcggccctgggctgcctggtcaaggactacttccccgaacctgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 8 - protein, 330 aa, artificial sequence [sequence omitted]

SEQ ID NO: 9 - DNA, 309 bp, artificial sequence
  synthesized
cgtacgaccaagggcccatcggtcttccccctggcaccctcctccaagagcacctctgggggcacagcggccctgggctgcctggtcaaggactacttccccgaacctgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 10 - protein, 103 aa, artificial sequence
  synthesized
RTTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 11 - DNA, 1347 bp, artificial sequence
  synthesized
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctgcagctagcaccaagggcccatcggtcttccccctggcaccctcctccaagagcacctctgggggcacagcggccctgggctgcctggtcaaggactacttccccgaacctgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 12 - protein, 449 aa, artificial sequence
  synthesized
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 636 bp, artificial sequence
  synthesized
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggatcccttccaggtttagtggcagtggatcagggacagattttactcttagcatcaacagtgtggagtctgaagatattgcagattattactgtcaacaaaataataactggccaaccacgttcggtgctgggaccaagctggagctgaaagtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatgc

SEQ ID NO: 14 - protein, 212 aa, artificial sequence
  synthesized
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 996 bp, artificial sequence
  synthesized
gtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 16 - protein, 332 aa, artificial sequence
  synthesized
VAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 321 bp, artificial sequence
  synthesized
gggcagccccgagaaccaaaggtgtacaccctgcccccatcccggaaggagctgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 18 - protein, 107 aa, artificial sequence
  synthesized
GQPREPKVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 19 - DNA, 321 bp, artificial sequence
  synthesized
gggcagccccgagaaccacaggtgtacaccctgcccccatcccgggatgagctgaccgagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctacagcgacctcaccgtggacaagagcaggtggcagcaggggaacgtcttctcatgctccgtgatgcatgaggctctgcacaaccactacacgcaggacagcctctccctgtctccgggtaaa

SEQ ID NO: 20 - protein, 107 aa, artificial sequence
  synthesized
GQPREPQVYTLPPSRDELTENQVSLTCLVKGFYPSDIAVEWESNGQPENNYDTTPPVLDSDGSFFLYSDLTVDKSRWQQGNVFSCSVMHEALHNHYTQDSLSLSPGK

SEQ ID NO: 21 - DNA, 321 bp, artificial sequence
  synthesized
gggcagccccgagaaccacaggtgtacaccctgcccccatcccgggatgagctgaccgagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctacagcgacctcaccgtggacaagagcaggtggcagcaggggaacgtcttctcatgctccgtgatgcatgaggctctgcacaaccactacacgcaggagagcctctccctgtctccgggtaaa

SEQ ID NO: 22 - protein, 107 aa, artificial sequence
  synthesized
GQPREPQVYTLPPSRDELTENQVSLTCLVKGFYPSDIAVEWESNGQPENNYDTTPPVLDSDGSFFLYSDLTVDKSRWQQGNVFSCSVMHEALHNHYTQESLSLSPGK

SEQ ID NO: 23 - DNA, 321 bp, artificial sequence
  synthesized
gggcagccccgagaaccaaaggtgtacaccctgcccccatcccggaaggagctgaccaagaaccaggtcagcctgacctgcctggtcaaaggcttctatcccagcgacatcgccgtggagtgggagagcaatgggcagccggagaacaactactgtaccacgcctcccgtgctgaagtccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 24 - protein, 107 aa, artificial sequence
  synthesized
GQPREPKVYTLPPSRKELTKNQVSLTCLVKGFYPSDIAVEWESNGQPENNYCTTPPVLKSDGSFFLYSKLTVDKSRWQQGNVFSCSVMHEALHNHYTQKSLSLSPGK

SEQ ID NO: 25 - DNA, 321 bp, artificial sequence
  synthesized
gggcagccccgagaaccacaggtgtacaccctgcccccatcccgggatgagctgaccgagaaccaggtcagcctgacctgcctggtcaaaggcttctatcccagcgacatcgccgtggagtgggagagcaatgggcagccggagaacaactacgacaccacgcctcccgtgctgtgctccgacggctccttcttcctctacagcgacctcaccgtggacaagagcaggtggcagcaggggaacgtcttctcatgctccgtgatgcatgaggctctgcacaaccactacacgcaggacagcctctccctgtctccgggtaaa

SEQ ID NO: 26 - protein, 107 aa, artificial sequence
  synthesized
GQPREPQVYTLPPSRDELTENQVSLTCLVKGFYPSDIAVEWESNGQPENNYDTTPPVLCSDGSFFLYSDLTVDKSRWQQGNVFSCSVMHEALHNHYTQDSLSLSPGK

SEQ ID NO: 27 - DNA, 321 bp, artificial sequence
  synthesized
gggcagccccgagaaccacaggtgtacaccctgcccccatcccgggatgagctgaccgagaaccaggtcagcctgacctgcctggtcaaaggcttctatcccagcgacatcgccgtggagtgggagagcaatgggcagccggagaacaactacgacaccacgcctcccgtgctgtgctccgacggctccttcttcctctacagcgacctcaccgtggacaagagcaggtggcagcaggggaacgtcttctcatgctccgtgatgcatgaggctctgcacaaccactacacgcaggagagcctctccctgtctccgggtaaa

SEQ ID NO: 28 - protein, 107 aa, artificial sequence
  synthesized
GQPREPQVYTLPPSRDELTENQVSLTCLVKGFYPSDIAVEWESNGQPENNYDTTPPVLCSDGSFFLYSDLTVDKSRWQQGNVFSCSVMHEALHNHYTQESLSLSPGK

SEQ ID NO: 29 - DNA, 1347 bp, artificial sequence
  synthesized
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctgcagctagcaccaagggcccatcggtcttccccctggcaccctcctccaagagcacctctgggggcacagcggccctgggctgcctggtcaaggactacttccccgaacctgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccaaaggtgtacaccctgcccccatcccggaaggagctgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 30 - protein, 449 aa, artificial sequence
  synthesized
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPKVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 31 - DNA, 996 bp, artificial sequence
  synthesized
gtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccgagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctacagcgacctcaccgtggacaagagcaggtggcagcaggggaacgtcttctcatgctccgtgatgcatgaggctctgcacaaccactacacgcaggacagcctctccctgtctccgggtaaa

SEQ ID NO: 32 - protein, 332 aa, artificial sequence
  synthesized
VAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYDTTPPVLDSDGSFFLYSDLTVDKSRWQQGNVFSCSVMHEALHNHYTQDSLSLSPGK

SEQ ID NO: 33 - DNA, 996 bp, artificial sequence
  synthesized
gtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccgagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctacagcgacctcaccgtggacaagagcaggtggcagcaggggaacgtcttctcatgctccgtgatgcatgaggctctgcacaaccactacacgcaggagagcctctccctgtctccgggtaaa

SEQ ID NO: 34 - protein, 332 aa, artificial sequence
  synthesized
VAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYDTTPPVLDSDGSFFLYSDLTVDKSRWQQGNVFSCSVMHEALHNHYTQESLSLSPGK

SEQ ID NO: 35 - DNA, 1347 bp, artificial sequence
  synthesized
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctgcagctagcaccaagggcccatcggtcttccccctggcaccctcctccaagagcacctctgggggcacagcggccctgggctgcctggtcaaggactacttccccgaacctgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccaaaggtgtacaccctgcccccatcccggaaggagctgaccaagaaccaggtcagcctgacctgcctggtcaaaggcttctatcccagcgacatcgccgtggagtgggagagcaatgggcagccggagaacaactactgtaccacgcctcccgtgctgaagtccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 36 - protein, 449 aa, artificial sequence
  synthesized
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPKVYTLPPSRKELTKNQVSLTCLVKGFYPSDIAVEWESNGQPENNYCTTPPVLKSDGSFFLYSKLTVDKSRWQQGNVFSCSVMHEALHNHYTQKSLSLSPGK

SEQ ID NO: 37 - DNA, 996 bp, artificial sequence
  synthesized
gtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccgagaaccaggtcagcctgacctgcctggtcaaaggcttctatcccagcgacatcgccgtggagtgggagagcaatgggcagccggagaacaactacgacaccacgcctcccgtgctgtgctccgacggctccttcttcctctacagcgacctcaccgtggacaagagcaggtggcagcaggggaacgtcttctcatgctccgtgatgcatgaggctctgcacaaccactacacgcaggacagcctctccctgtctccgggtaaa

SEQ ID NO: 38 - protein, 332 aa, artificial sequence
  synthesized
VAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYDTTPPVLCSDGSFFLYSDLTVDKSRWQQGNVFSCSVMHEALHNHYTQDSLSLSPGK

SEQ ID NO: 39 - DNA, 996 bp, artificial sequence
  synthesized
gtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccgagaaccaggtcagcctgacctgcctggtcaaaggcttctatcccagcgacatcgccgtggagtgggagagcaatgggcagccggagaacaactacgacaccacgcctcccgtgctgtgctccgacggctccttcttcctctacagcgacctcaccgtggacaagagcaggtggcagcaggggaacgtcttctcatgctccgtgatgcatgaggctctgcacaaccactacacgcaggagagcctctccctgtctccgggtaaa

SEQ ID NO: 40 - protein, 332 aa, artificial sequence
  synthesized
VAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYDTTPPVLCSDGSFFLYSDLTVDKSRWQQGNVFSCSVMHEALHNHYTQESLSLSPGK

SEQ ID NO: 41 - DNA, 57 bp, artificial sequence
  synthesized
atgggatggtcatgtatcatcctttttctagtagcaactgcaaccggtgtacattcc